SEQUENCE LISTING (4 sequences)

SEQ ID NO: 1 - DNA (cDNA to mRNA), 1302 bp; single-stranded, linear
  CDS 2..145
  repeat_region 981..1279
  hypothetical: NO
  anti-sense: NO
  fragment type: C-terminal
gaattcggcacgagctagagcaagcgcggccccgcggcccggagccatgctgaggagctgcgccgcgcgcctccgcacgctgggggctctgtgccggccgccagtaggccggcgcctgccggaagcgacccgcgacccgagctgaggtcattttcttctgaggaagtcattcttaaggactgttctgtccccaaccccagctggaacaaggacctaagactgctctttgaccagtttatgaagaaatgtgaagatggctcctggaaacgtttgccttcatataaacgtacacctactgaatggattcaagacttcaaaacccattttcttgacccaaagcttatgaaagaagaacaaatgtcacaggcccagctcttcaccagaagctttgatgatggcctgggctttgaatacgtgatgttctacaatgacattgagaaaaggatggtttgcttatttcaaggaggcccttacctggaagaccacctggattcattcatggaggtgccattgcaaccatgattgatgctactgttggtatgtgtgcaatgatggctgggggaatcgtcatgactgccaatctcaacatcaattatccccgacctatccctctttgttctgttgttatgataaatagccaacttgataaagttgaaggaaggaaattttttgtttcctgtaatgttcagagtgttgatgagaagaccctatactcagaggcgacaagcttattataaagtcgaatcctgctaaaagtcttgatcgataaagagtcgtcggtgaactccatctcattctcgcccctccagaagaaggcagttgtcccccaaatactctgctccctcactgctgaatcctgtagggagaagcctgccaacagtgaccttccgaaacagccttctgaatacaaagaggattcagtttccatcttctcaactttttaacacagaaacacttcctgcgagactatcgacaactctcgggccaggcgctgtggctcacacctgtaatcccagcactttaggaggccgaggcaggcggattgcctgagctcaggagttgaagatcagtctgggcaacacgatgaaactccgtctctactaaaatacaaaaaattatccaggcatggtggcgtacgcctgtagtcccagctactcaggaggctgaggcaggagaatcgcttgaacccaggaggaagaggttgcagtgagccaagatcatgccacatcactccaacctgggcaacagaacaagaacccatctcaaacaaacaacaaacaaaaaaaaaaaaaaaaactcgag

SEQ ID NO: 2 - protein, 47 aa [sequence omitted]

SEQ ID NO: 3 - DNA (cDNA to mRNA), 2610 bp, Homo sapiens; single-stranded, linear
  CDS 199..1641
  mat_peptide 199..1641
  hypothetical: NO
  anti-sense: NO
  immediate source clone: Human RAC alpha
atcctgggacagggcacagggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctacagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattct

SEQ ID NO: 4 - protein, 480 aa
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

(19) United States
(12) Patent Application Publication
(10) Pub. No.: US 2002/0127214 A1
(43) Pub. Date: Sep. 12, 2002

(54) RAC-protein kinase as therapeutic agent or in diagnostics

(76) Inventor: Brian Arthur Hemmings, Bettingen (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/970,000

(22) Filed: Oct. 3, 2001

Related U.S. Application Data
(63) Continuation of application No. 09/068,702, filed on May 13, 1998, now abandoned.
Application No. 09/068,702 is a 371 of international application No. PCT/EP96/04810, filed on Nov. 5, 1996.

(30) Foreign Application Priority Data
Nov. 16, 1995 (GB) 9523379.7
Dec. 15, 1995 (GB) 9525704.4

(51) Int. Cl. A61K 38/52; C12N 9/12

(52) U.S. Cl. 424/94.5; 435/194

(57) ABSTRACT

The invention concerns RAC-PK protein kinase and fragments thereof, as well as activators and inhibitors of RAC-PK protein kinase for use as medicaments, particularly in the treatment of diseases concerned with abnormalities in processes modulated by insulin, such as cellular proliferation, insulin deficiency and/or excess blood sugar levels. Moreover, the invention provides RAC-PK protein kinase for use in screening potential mimics or modulators thereof. The invention further provides a screening kit comprising the RAC-PK as an active principle, and a method for screening compounds which are candidate mimics or modulators of RAC-PK activity comprising detecting specific interactions between the candidate compounds and RAC-PK.

This is a continuation of Ser. No. 09/068,702, May 13, 1998, pending, which is a 371 of PCT/EP96/048101, May 11, 1996.

The present invention concerns the use of RAC protein kinase (RAC-PK) as a therapeutic agent, as a ligand for screening molecules for a possible interaction with RAC-PK and to a method for identifying molecules involved in signal transduction.

Protein phosphorylation and dephosphorylation are fundamental processes for the regulation of cellular functions. Protein phosphorylation is prominently involved in signal transduction, where extracellular signals are propagated and amplified by a cascade of protein phosphorylation and dephosphorylation. Two of the best characterised signal transduction pathways involve the c-AMP-dependant protein kinase (PKA) and protein kinase C (PKC). Each pathway uses a different second messenger molecule to activate the protein kinase, which, in turn, phosphorylates specific target molecules.

A novel subfamily of serine/threonine kinases has been recently identified and cloned, termed herein the RAC kinases (RAC-PK; Jones, et al. (1991) Proc. Natl. Acad. Sci. USA 88, 4171-4175; Jones, et al. (1991) Cell Regulation 2, 1001-1009), but also known as PKB or Akt. RAC kinases have been identified in two closely related isoforms, RACα and RACβ, which share 90% homology at the gene sequence. Mouse RACα (c-akt) is the cellular homologue of the viral oncogene v-akt, generated by fusion of the Gag protein from the AKT8 retrovirus to the N-terminus of murine c-akt. Human RACβ is found to be involved in approximately 10% of ovarian carcinomas, suggesting an involvement of RAC kinases in cell growth regulation.

RAC kinases contain an amino-terminal pleckstrin homology (PH) domain [Haslam, et al. (1993) Nature 363, 309-310]. The PH domain was originally identified as an internal repeat, present at the amino and carboxy-termini of pleckstrin, a 47 kDa protein which is the major PKC substrate in activated platelets [Tyers, et al. (1988) Nature 333,470-473]. The superfamily of PH domain containing molecules consists of over 90 members, including serine/threonine protein kinases (RAC, Nrk, βARK, PKCμ), tyrosine protein kinases (Btk, Tec, Itk), GTPase regulators (ras-GAP, ras-GRF, Vav, SOS, BCR), all known mammalian phospholipase Cs, cytoskeletal proteins (β-spectrin, AFAP-110, syntrophin), “adapter” proteins (GRB-7, 3BP2) and kinase substrates (pleckstrin, IRS-1).

While the PH domain structure has been solved for β-spectrin, dynamin and pleckstrin's amino-terminal domain, its precise function remains unclear. The presence of PH domains in many signalling and cytoskeletal proteins implicates it in mediating protein-protein and membrane interactions. Indeed, the PH domain of the β-adrenergic receptor kinase (βARK) appears partly responsible for its binding to the βγ-subunits of the heterotrimeric G-proteins associated with the β-adrenergic receptor, while the PH domain of the Bruton's tyrosine kinase (Btk) appears to mediate an interaction with PKC. Several PH domains have been shown to be able to bind phosphatidyl-inositol-4-5-bisphosphate in vitro, although weakly.

IMPDH is a highly conserved enzyme (41% amino acid identity between bacterial and mammalian sequences) involved in the rate-limiting step of guanine biosynthesis. In mammals there are two isoforms, 84% identical, called type I and type II which are differentially expressed [Natsumeda, et al. (1990) J. Biol. Chem. 265, 5292-5295]. Type I is constitutively expressed at low levels while the type II mRNA and protein levels increase during cellular proliferation. IMPDH activity levels are also elevated during rapid proliferation in many cells [Collart, F. R., and Huberman, E. (1988) J. Biol. Chem. 263, 15769-15772].

By measuring the metabolic fluxes, the proliferative index of intact cancer cells has been shown to be linked with the preferential channelling of IMP into guanylate biosynthesis. Inhibition of cellular IMPDH activity results in an abrupt cessation of DNA synthesis and a cell-cycle block at the G₁-S interface. The specific inhibition of IMPDH by tiazofurin and the subsequent decline in the GTP pool, results in the down regulation of the G-protein ras, which is involved in many signal transduction pathways leading to cellular proliferation (for review see Avruch, et al. (1994) Trends Biochem. Sci. 19, 279-283).

Interestingly, p53 has been implicated in regulating IMPDH activity levels [Sherley, J. L. (1991) J. Biol. Chem. 266, 24815-24828]. Here a moderate over-expression of p53 (3-6 fold) induces a profound growth arrest which is rescued by purine nucleotide precursors. Indeed, the p53 over-expression induces a specific block in IMP to XMP conversion, and a diminished activity level of IMPDH. The p53 block does not affect the rate of RNA synthesis, nor is the phenotype rescued by deoxynucleotides indicating that a lack of precursors for DNA synthesis is also not the cause of the block. It would seem most likely that this effect is mediated through a down regulation of the GTP pool required by G-proteins such as ras.

The above observations suggest that IMPDH type II is primarily involved in producing XMP which is channelled into the GTP pool which is crucial for the regulation of G-proteins involved in signal transduction such as ras. It may be that the type I enzyme provides a basal level of XMP that is channelled into the GTP/dGTP pools required for RNA and DNA synthesis. Changes in IMPDH type II activity would alter the GTP/GDP ratio by specifically altering the GTP component which could greatly affect ras signalling pathways as ras is sensitive to small changes in the GTP/GDP ratio.

Glycogen synthase kinase-3 (GSK3) is implicated in the control of several processes important for mammalian cell physiology, including glycogen metabolism and the control of protein synthesis by insulin, as well as the modulation of activity of several transcription factors, such as AP-1 and CREB. GSK3 is inhibited in vitro by serine phosphorylation caused by MAP kinase and p70^{S6K}, kinases which lie on distinct insulin-stimulated signalling pathways.

GSK3 is responsible for serine phosphorylation in glycogen synthase, whose dephosphorylation underlies the stimulation of glycogen synthesis by muscle. Thus, GSK3 inactivates glycogen synthase, resulting in an increase in blood sugar levels. Insulin inhibits the action of GSK3, which, in combination with the concomitant activation of phosphatases which dephosphorylate glycogen synthase, leads to the activation of glycogen synthase and the lowering of blood sugar levels.

We have now determined that RAC-PKis directly involved in the inactivation of GSK3 and the activation of IMPDH.

We have used the yeast two-hybrid system [Fields, S. and Song, O -K. (1989) Nature 340, 245-246; Chien, et al. (1991) Proc. Natl. Acad. Sci. U.S.A. 88, 9578-9582] to determine if RAC-PK could function by forming specific interactions with other proteins. We have identified RAC-PK as interacting with human inosine-5′ monophosphate dehydrogenase (IMPDH) type II, and with a novel protein termed RAC-PK Carboxy-Terminal Binding Protein (CTBP). RAC-PK stimulates IMPDH Type II activity. In conjunction with the known role of IMPDH in GTP biosynthesis our findings suggest a role for RAC-PK in the regulation of cell proliferation.

Moreover, using a peptide derived from GSK3 and GSK3 itself, we have been able to show that RAC-PK interacts with, phosphorylates and inactivates GSK3. This implicates RAC-PK in the regulation of insulin-dependent signalling pathways, which control cellular proliferation. Taken together, these results suggest a major involvement for RAC-PK in the control of insulin action.

### SUMMARY OF THE INVENTION

According to the invention, we provide RAC-PK protein kinase and fragments thereof, as well as activators and inhibitors of RAC-PK protein kinase for use as medicaments, particularly in the treatment of diseases concerned with abnormalities in processes modulated by insulin, such as cellular proliferation, insulin deficiency and/or excess blood sugar levels. Moreover, the invention provides RAC-PK protein kinase for use in screening potential mimics or modulators thereof. The invention further provides a screening kit comprising the RAC-PK as an active principle, and a method for screening compounds which are candidate mimics or modulators of RAC-PK activity comprising detecting specific interactions between the candidate compounds and RAC-PK.

### DETAILED DESCRIPTION OF THE INVENTION

In a first aspect, the invention provides RAC protein kinase or a fragment thereof, or a modulator thereof except vanadate and wortmannin, for use as a medicament.

Vanadate, which term as used herein includes various forms thereof such as ortho- and metavanadate, pervanadate and other related vanadium ions, is known as a therapeutic agent in the treatment of diabetes. See, for example, U.S. Pat. No. 5,421,125, European Patent Application 0 521 787, European Patent Application 0 264 278 and European Patent Application 0 245 979. In UK Patent Application 9525702.8 (Ciba-Geigy AG), filed Dec. 15, 1995, it is disclosed that vanadate is a potent activator of kinases of the insulin-stimulated signalling pathways and of RAC-PK in particular. Accordingly vanadate exerts its therapeutic effect on diabetes by stimulating RAC, which phosphorylates GSK3 and deactivates it, leading to a lowering in blood sugar levels. RAC-PK and activators thereof are therefore useful in the treatment of diabetes and other diseases where blood sugar levels are excessive. Conversely, inhibitors of RAC-PK such as wortmannin and okadaic acid are useful in the treatment of diseases involving insufficiency in blood sugar levels.

In the present invention, RAC protein kinase may be any isoform of RAC kinase as described in the literature, from any species. Human RAC-PK is preferred. Human RAC-PKα is represented in SEQ ID No. 3. Domains of RAC kinase are the individual functional portions thereof, such as the PH domain, the catalytic domain and the C-terminal domain. Fragments of RAC, which include domains of RAC, are functionally active portions of the RAC kinase protein which may be used in the present invention in place of RAC. Fragments of RAC-PK are preferably the domains thereof, advantageously the PH domain, the catalytic domain and the C-terminal domain. In each case, the terminology used embraces mutants and derivatives of RAC-PK and its fragments which can be created or derived from the naturally-occurring protein according to available technology. For instance, nucleic acids encoding RAC-PK may be mutated without affecting the nature of the peptide encoded thereby, according to the degeneracy of the amino acid code. Moreover, conservative amino acid substitutions may be made in RAC-PK or a fragment thereof, substantially without altering its function. Further additions, deletions and/or substitutions which improve or otherwise alter the function of RAC-PK or a fragment thereof are envisaged and included within the scope of the invention.

The invention also provides the use of RAC protein kinase or a fragment thereof, or a modulator of RAC protein kinase activity, except vanadate, for the preparation of a medicament for use in the treatment of diseases involving an anomaly in blood sugar levels, such as diabetes.

Moreover, the invention provides the use of RAC protein kinase or a fragment thereof, or a modulator of RAC protein kinase activity, except wortmannin, for the preparation of a medicament for use in the treatment of abnormalities in cellular proliferation.

The antibiotic wortmannin, which is known to inhibit phosphatidylinositol 3-OH kinase (PI-3K) activation, targets signal transduction and indirectly inactivates inter alia RAC, possibly via PI-3K. Wortmannin has been indicated in the treatment of neoplastic conditions.

However, the broad involvement of the various isoforms of RAC-PK in mitogenic signal transduction as well as insulin-dependent signalling has hitherto not been known. We have now shown that RAC-PK is involved in the regulation of both GSK3 and IMPDH, a factor involved in growth control. It can be concluded, therefore, that RAC-PK plays a central role in growth control.

Therapeutic agents according to the invention may be formulated conventionally, according to the type of agent. Where the agent is a salt, such as vanadate, it is conveniently formulated in aqueous solution at neutral pH and administered orally at room temperature. In the case of a peptide medicament, such as RAC-PK itself, more elaborate delivery techniques, such as liposomal delivery, may be required in order to introduce the peptide into target cells. Delivery systems for peptide therapeutics are documented in the art.

The identification of RAC-PK as a major mediator in growth control permits the design of screening systems to identify putative therapeutic agents for use in treating anomalies of growth control. Thus, in a second aspect of the invention there is provided a method for screening potential modulators of intracellular signalling comprising the steps of:

(a) incubating RAC-PK or a fragment thereof with the compound to be screened; and

(b) detecting interaction between the compound and RAC.

The screening may be carried out using complete RAC kinase or a fragment thereof. In particular, it has been shown that the PH domain of RAC kinase is important in mediating many of its effects, as set out, for example, in UK patent application 9525703.6 (Ciba-Geigy AG), filed Dec. 15, 1995. Moreover, as disclosed hereinbelow, RAC-PK interacts with IMPDH via the PH domain. The interaction is not observable in the yeast two-hybrid system if complete RAC-PK is used, although in vitro binding of RAC-PK to IMPDH occurs.

Interactions also occur between other fragments of RAC-PK and its physiological targets and regulators. For example, GSK3 binds to RAC-PK via the catalytic domain, as evidenced by the phosphorylation of GSK3 by RAC. CTBP, on the other hand, does not bind the catalytic or PH domains but binds specifically to the carboxy terminal domain of RAC.

Preferably, therefore, the invention includes incubating the compound to be screened with a fragment of RAC, which is advantageously the PH domain, the catalytic domain or the carboxy terminal domain.

RAC-PK fragments for use in the method of the present invention may be in the form of isolated fragments, or in the form of the fragment complexed with further polypeptides. For example, in the case of the two-hybrid system, the fragment is complexed to a DNA binding or transcriptional activation domain derived from another protein, such as the yeast activator GAL4.

Moreover, the RAC-PK used in the method of the invention may be in the form of a mutant thereof, for example a constitutively activated kinase. An important activating residue is T308, present in the so-called T-loop between subdomains 7 and 8 of the kinase. A general guide to kinase structure is given in Woodgett (1994), Protein Kinases, IRL Press, UK. Substitution of T308 with aspartic acid results in a clear increase in basal activity of the kinase, which however retains a potential for further activation. The invention therefore provides a RAC kinase protein in which Thr3O8 has been mutated to Asp.

Preferably, Ser473 is additionally mutated to Asp. Phosphorylation of this residue is required for full activation of RAC-PK in vivo, and the T308/S473 double mutant (both residues converted to Asp) shows a constitutive activity 18-fold higher than native RAC-PK. The double mutant is not susceptible to further activation.

The mutations may be carried out by means of any suitable technique. Preferred, however, is in vitro site-directed mutagenesis of a nucleotide sequence encoding RAC and subsequent expression of RAC in a recombinant DNA expression system. This method is an in vitro mutagenesis procedure by which a defined site within a region of cloned DNA can be altered (cf. the review articles of M. J. Zoller and M. Smith, Methods Enzymol. (1983), 100, 468; D. Botstein and D. Shortle, Science (1985), 229, 1193). Methods for site-directed mutagenesis are well known to those of skill in the art, as exemplified by Sambrook (1989): Molecular Cloning—A Laboratory Manual, Cold Spring Harbor, N.Y., USA, and the number of commercially available in vitro mutagenesis kits.

The compound to be screened may be present in essentially pure, uncomplexed form, or may be complexed with chemical groups or further polypeptides. In the case of the two hybrid system, it is complexed to a DNA binding or transcriptional activation domain, in order to complement the PH domain.

Isolated PH domain for use in the present invention may be prepared as set forth in our UK patent application 9525705.1 (Ciba-Geigy AG), filed Dec. 15, 1995. Where a small quantity of PH domain suffices, however, PH domain may be obtained by expressing a nucleic acid sequence encoding it in bacterial cell culture in the form of a fusion protein which is subsequently cleaved according to techniques known in the art. For example, amino acids 1-131 of RAC, which encode the PH domain; may be expressed as a fusion protein, advantageously with glutathione-S-transferase (GST), subsequently cleaving the fusion protein with thrombin and isolating the domain by protein purification techniques such as FPLC. This method gives a relatively small yield of pure soluble PH domain.

Carboxy and kinase domains are likewise advantageously synthesised as fusion proteins, for instance as GST fusions.

RAC-PK or a fragment thereof for use in the present invention may be prepared as set forth in UK patent application 9525702.8. Alternatively, RAC-PK may be expressed in recombinant cell culture. Baculovirus vectors, specifically intended for insect cell culture, are especially preferred and are widely obtainable commercially (e.g. from Invitrogen and Clontech). Other virus vectors capable of infecting insect cells are known, such as Sindbis virus (Hahn et al., (1992) PNAS (USA) 89, 2679-2683). The baculovirus vector of choice (reviewed by Miller (1988) Ann. Rev. Microbiol. 42, 177-199) is Autographa californica multiple nuclear polyhedrosis virus, AcMNPV.

Typically, the heterologous gene replaces at least in part the polyhedrin gene of AcMNPV, since polyhedrin is not required for virus production. In order to insert the heterologous gene, a transfer vector is advantageously used. Transfer vectors are prepared in E. coli hosts and the DNA insert is then transferred to AcMNPV by a process of homologous recombination. Baculovirus techniques useful in the present invention are standard and well known in the art, and are reviewed in O'Reilly et al., (1 994) Baculovirus expression vectors; A laboratory manual, Oxford University Press Inc., NY, USA, as well as in literature published by suppliers of commercial baculovirus kits (e.g. Pharmingen).

Incubation conditions will vary according to the precise method used to detect the interaction between the PH domain and the screened compound. In the case of transcription activation detection systems such as the yeast two-hybrid system, incubation conditions are suitable for gene transcription, such as those prevailing inside a living cell. Other detection systems, however, will require different incubation conditions. For example, if the detection of interaction is based on relative affinity in a chromatographic assay, for example as is known in affinity chromatography, conditions will be adjusted to promote binding and then gradually altered, such that the point at which the screened compound no longer binds to the RAC-PK PH domain may be determined.

The detection method may employ the natural fluorescence of Trp²² in the RAC-PK PH domain, which is inhibited by certain interactions with the domain, as set forth in UK patent application 9525703.6. Briefly, fluorescence of the amino-terminal Trp residue in the PH domains of certain PH domain containing proteins may be detected by exciting the molecule to fluoresce at the appropriate frequency and monitoring the emission. The N-terminal Trp²² of RAC, for example, fluoresces at 345 nm when excited at 290 nm. Techniques for monitoring protein fluorescence are widely known in the art. We have shown that the PH domain of RAC-PK binds phospholipid with high affinity, which suggests that RAC-PK may be membrane-bound in vivo via the PH domain. Binding of phospholipid to the RAC-PK PH domain quenches the natural fluorescence of the N-terminal tryptophan at position 22 (Trp²²). Interaction of the PH domain with the cell membrane is believed to be important for the stable interaction of RAC-PK with membrane bound partners in signalling pathways, such that disruption of this interaction will lead to modulation of the signalling effect through the dissociation of the signalling molecule from the cell membrane. The modulation could be either down-regulating, for example if the otherwise stable interaction of the molecule with membrane-bound partners is a stimulatory interaction, or up-regulating, in the event that the interaction is an inhibitory interaction. Accordingly, a compound which is a candidate modulator of signal response may be screened for by means of a method comprising the steps of:

(a) incubating the compound with the PH domain of a signalling molecule which is capable of fluorescing;

(b) determining the phospholipid-induced modulation in the fluorescence of the PH domain, an alteration of the fluorescence in the presence of the compound being indicative of a functional interaction between the compound and the PH domain.

In this case, the incubation conditions will be adjusted to facilitate the detection of fluorescence at 345 nm when the PH domain is excited at a frequency of 290 nm.

Incubation according to the invention may be achieved by a number of means, but the basic requirement is for RAC-PK or a fragment thereof and the screened compound to be able to come into contact with each other. This may be achieved by admixing RAC-PK or a fragment thereof and the compound, or by producing them in situ, such as by expression of nucleic acids encoding them. Where the RAC-PK or RAC-PK fragment and/or the compound are in the form of fusions with other polypeptides, they may be expressed as such in situ.

Preferably, the method of the invention is based on a two-hybrid system. Such systems detect specific protein:protein interactions by exploiting transcriptional activators having separable DNA-binding and transcription activating domains, such as the yeast GAL4 activator. A reporter gene is operatively linked to an element responsive to the transcriptional activator being used, and exposed to RAC-PK or a fragment thereof and the compound to be screened, one of which is complexed to the transcription activating domain of the transcriptional activator and the other of which is joined to the DNA binding domain thereof. If there is a specific interaction between RAC-PK or a fragment thereof and the compound, the DNA binding and transcription activating domains of the transcriptional activator will be brought into juxtaposition and transcription from the reporter gene will be activated.

Alternatively, the detection may be based on observed binding between RAC-PK or a fragment thereof, such as its PH domain or its catalytic domain, and the screened compound, or a fragment thereof. For example, the interaction between RAC-PK and the insulin mediator GSK3 is detected hereinbelow by monitoring the interaction of a peptide surrounding the major phosphorylation site of GSK3 known to be responsible for its inactivation with RAC kinase. In a similar manner, the involvement of RAC-PK on the activation or inactivation of a particular compound may be screened for by monitoring the interaction of a portion thereof known to be involved in modulation events with RAC.

RAC-PK or a fragment thereof may be used to screen for compounds which bind thereto by incubating it with the compound to be screened and subsequently “pulling down” RAC-PK complexes with a RAC-specific antibody. Antibodies suitable for immunoprecipitation or immuno-affinity chromatography may be prepared according to conventional techniques, known to those of ordinary skill in the art, and may be monoclonal or polyclonal in nature. For example, see Lane, et al., (1992) EMBO J., 11, 1743-1749. After the RAC-compound complex has been isolated by affinity, the compound may be dissociated from the RAC-PK antibody and characterised by conventional techniques.

The interaction of RAC-PK or a fragment thereof with the screened compound may also be observed indirectly. For example, an inhibitor or activator of RAC-PK function may be detected by observing the effects of RAC-PK on a substrate in the presence or absence of the compound.

The activity of RAC-PK or the catalytic domain thereof may be assessed by means of a kinase activity assay, employing a substrate for the kinase. For example, myelin basic protein may be used, in accordance with established assay procedures. Physiological substrates, such as GSK3, may also be used. Alternatively, RAC-PK activity may be assessed by determining the degree of activating phosphorylation of RAC-PK itself Advantageously, phosphorylation on residues normally implicated in kinase activation is assessed. RAC-PK, as disclosed in UK patent application 9525702.8, is preferentially activated by phosphorylation at serine and threonine residues.

The assay of the invention may be used to measure the direct effect of the candidate compound on RAC, or it may be used to determine the effect of the compound on a kinase acting upstream thereof in a signalling pathway. In the latter situation, RAC-PK acts as a substrate for the upstream kinase and the activity of the upstream kinase is assessed by determining the phosphporylation state or the activity of RAC-PK.

In order to obtain a meaningful result, the activity of RAC-PK exposed to the candidate immunosuppressive or antiproliferative agent should be compared to the activity of RAC-PK not exposed to the agent, a modulation of RAC kinase activity being indicative of potential as a modulator of cell proliferation and/or insulin signal transduction.

Promising compounds may then be further assessed by determining the properties thereof directly, for instance by means of a cell proliferation assay. Such an assay preferably involves physical determination of proliferation in cells which have been subjected to kinase activation by a phosphatase inhibitor, exposed to the candidate RAC-PK modulator and optionally subsequently stimulated with a mitogen, such as a growth factor, IL-2 or PMA. More simply, the assay may involve exposure of unstimulated cells to the candidate modulator, followed by stimulation with a phosphatase inhibitor.

The invention further comprises the use of RAC-PK or a fragment thereof in a screening system. The screening system is preferably used to screen for compounds which are modulators of insulin activity, particularly where that activity is related to glycogen metabolism or cell proliferation.

Kits useful for screening such compounds may be prepared, and will comprise essentially RAC-PK or a fragment thereof together with means for detecting an interaction between RAC-PK and the screened compound. Preferably, therefore, the screening kit comprises one of the detection systems set forth hereinbefore.

RAC-PK for use in kits according to the invention may be provided in the form of a protein, for example in solution, suspension or lyophilised, or in the form of a nucleic acid sequence permitting the production of RAC-PK or a fragment thereof in an expression system, optionally in situ. Preferably, the nucleic acid encoding RAC-PK or a fragment thereof encodes it in the form of a fusion protein, for example a GST fusion.

In a still further embodiment, the invention provides a compound which interacts directly or indirectly with RAC-PK or a fragment thereof. In the case of indirectly acting compounds, agents such as insulin and wortmannin are excluded. Such a compound may be inorganic or organic, for example an antibiotic, and is preferably a proteinaceous compound involved in intracellular signalling. For example, the compound may be CTBP (SEQ ID Nos. 1 and 2).

Compounds according to the invention may be identified by screening using the techniques described hereinbefore, and prepared by extraction from natural sources according to established procedures, or by synthesis, especially in the case of low molecular weight chemical compounds. Proteinaceous compounds may be prepared by expression in recombinant expression systems, for example a baculovirus system as described hereinbefore or in a bacterial system, for example as described in UK patent application 9525705.1. Proteinaceous compounds are mainly useful for research into the function of signalling pathways, although they may have a therapeutic application.

Low molecular weight compounds, on the other hand, are preferably produced by chemical synthesis according to established procedures. They are primarily indicated as therapeutic agents. Low molecular weight compounds and organic compounds in general may be useful as insulin mimics or antiproliferative agents.

The invention is further described, for the purposes of illustration only, in the following examples.

### EXAMPLE 1

### Specific Interaction of RAC-PK with IMPDH

(a) Bacterial and Yeast Strains

All yeast strains and plasmids for two-hybrid experiments are obtained from Clontech (Palo Alto, Calif.) as components of the MATCHMAKER Two Hybrid System or from Dr. D. Nathans (Howard Hughes Medical Institute, Baltimore, Md.). Yeast strains SFY526 (MATa, ura3-52, his3-200, ade2-101, lys2-801, trp1-901, leu2-3, 112, can^{r}, gal4-542, gal80-538, URA3::GAL1-lacZ), HF7c (MATa, ura3-52, his3-200, lys2-801, ade2-101, trp1-901, leu2-3, 112, gal4-542, gal80-538, LYS2::GAL1-HIS3, URA3::(GAL4 17-mer)₃-CYC1-lacZ) and PCY2 [Chevray and Nathans, 1992] (MATα, his3-200, ade2-101, lys2-801, trp1-63, leu2-3, gal4-542, gal80-538, URA3::GAL1-lacZ) are used to assay for protein-protein interactions. Yeast strain HF7c is used for library screening. SFY526 and PCY2 have the upstream activating sequence and TATA sequence of the GAL1 promoter fused to the lacZ gene. In HF7c, HIS3 is fused to a GAL1 promoter sequence and LacZ is fused to three copies of a 17-mer GAL4 consensus sequence plus the TATA sequence of the CYC1 promoter. Both HIS3 and LacZ are responsive to GAL4 activation. Yeast techniques including transformation are performed according to the instructions in the MATCHMAKER Two Hybrid System and as described [Ausubel, et al. (1994) Current Protocols in Molecular Biology. John Wiley and Sons, New York, N.Y.]. The bacterial strains XL1 -blue (Statagene) and JM109 are employed in the cloning of plasmids and the production of GST fusion proteins. The bacterial strains JM109(DE3), BL21(DE3)pLysS and BL21(DE3)pLysE (Invitrogen) are used for the production of (HIS)₆-tagged proteins. General molecular biological techniques are performed as previously described [Sambrook, et al. (1989) Molecular Coning: A Laboratory Manual., 2nd Ed., Cold Spring Harbor Laboratory, Cold Spring Harbor, N.Y.; Davis, et al. (1986) Basic Methods in Molecular Biology. Elsevier Science Publishing Co., New York, N.Y.].

(b) Plasmid Construction

Yeast vector plasmids containing the GAL4 DNA binding domain (amino acids 1-147, pGBT9) and the GAL4 activation domain (amino acids 768-881, pGAD424) as well as the control plasmids pCL1 (full-length GAL4 gene), pVA3 (p53 gene), pTD1 (SV40 large T antigen), and pLAM5′ (human lamin C gene) are from Clontech. The yeast vector pPC62, containing the GAL4 DNA binding domain, is from Dr. D. Nathans. The glutathione-S-transferase (GST) fusion vector pGEX-2T is from Pharmacia. The baculovirus transfer vector (pVL1392) and the (HIS)₆-tag vector (pRSET-A) are from Invitrogen. pGBT-PH127, pGBT-PH150, pGBT-PHI-III, and pGBT-PHIII-VI contain in-frame fusions of amino acids 1-127, 1-150, 1-47, 47-127 of the human RACα PH domain, respectively, with the GAL4 DNA binding domain. They are constructed by subcloning PCR fragments generated with specific oligonucleotides into the EcoRI-BamHI sites of pGBT9. pGEX-PH131, pGEX-PH-KIN, pGEX-PH-KIN-CT, pGEX-KIN-CT, pGEX-KIN, and pGEX-CT contained in-frame fusions of amino acids 1-131, 1-411, 1-480, 147-480, 147-411, 411-480 of human RACα, respectively, with GST. They are constructed by subcloning PCR fragments generated with specific oligonucleotides into the BamHI-EcoRI sites of pGEX-2T. pGBT-PH-KIN, pGBT-PH-KIN-CT, pGBT-KIN-CT, pGBT-KIN, and pGBT-CT contain in-frame fusions of amino acids 1-411, 1-480, 147-480, 147-411, 411-480 of human RACα, respectively, with the GAL4 DNA binding domain. They are constructed by subcloning the appropriate BamHI-EcoRI fragments from the corresponding pGEX constructs into the PstI-XbaI sites of pPC62 using PstI-BamHI and EcoRI-XbaI adapters. The XhoI-XbaI fragments from the resultant pPC62 plasmids are then isolated and subcloned into the XhoI-EcoRI sites of pGBT9 using a XbaI-EcoRI adapter. pGAD-IMPDH1-481, pGAD-IMPDH1-427, pGAD-IMPDH1-325, pGAD-IMPDH28-514, pGAD-IMPDH70-514, pGAD-IMPDH1-40, and pGAD-IMPDH428-514 contain in-frame fusions of amino acids 1-481, 1-427, 1-325, 28-514, 70-514, 1-40, 428-514 of human IMPDH type II, respectively, with the GAL4 activation domain. They are constructed by subcloning PCR fragments generated with specific oligonucleotides into the BamHI-SalI sites of pGAD424. pGEX-IMPDH contains an in-frame fusion of the complete human IMPDH type II with GST. It is constructed by subcloning the SmaI-XhoI IMPDH fragment from pGADGH-IMPDH into the SmaI site of pGEX-2T using a XhoI-SmaI adapter. pVL1392-hRACα contained the EcoRI fragment from WI38xRAC71 [Jones, et al. (1991) Proc. Natl. Acad. Sci. U.S.A. 88, 4171-4175] encompassing the full coding region of human RACα. pRSET-PHQKKK contains an in-frame fusion of amino acids 1-116 of human RACα with an amino-terminal (HIS)₆-tag, and the addition of three lysines at the carboxy terminus. It is constructed by subcloning an NdeI-PfIMI fragment from pRK-RAC [Jones, et al. (1991) Proc. Natl. Acad. Sci. U.S.A. 88, 4171-4175] into the BamHI-EcoRI sites of pRSET-A using BamHI-NdeI and PfIMI-EcoRI adapters. All plasmid constructions are confirmed by restriction fragment analysis and sequencing.

(c) Library Screening

To determine if RAC's PH domain could interact with other proteins we fuse it to the GAL4 DNA binding domain and screen a HeLa cDNA library fused to the GAL4 transcriptional activation domain in the yeast reporter strain HF7c. The human HeLa S3 MATCHMAKER cDNA library is purchased from Clontech. pGBT-PH127 is transformed into HF7c with and without the control plasmids (pGAD424, pCL1, pTD1). Colonies from this transformation are tested for HIS3 and LacZ expression to confirm that the PH domain alone does not activate transcription. The HF7c transformant containing just pGBT-PH127 is then transformed with enough of the HeLa S3 cDNA library inserted into the two-hybrid activation vector pGADGH to produce 1.0×10⁶ yeast Leu⁺/Trp⁺ transformants. Doubly transformed cells are plated onto Leu⁻, Trp⁻, His⁻ plates and incubated at 30° C. for 3-8 days. Positive colonies are picked, restreaked onto triple minus plates and assayed for LacZ activity by the filter assay. Library clones that are His⁺ and LacZ⁺ are then cured of the pGBT-PH127 plasmid and tested again for His auxotrophy and LacZ activity. Cured clones that are negative in both assays are then mated to transformants of PCY2 containing either pGBT9, pGBT-PH127, pLAM5′ or pTD1. The activation clones corresponding to the diploids which become positive for both His auxotrophy and LacZ activity only in the presence of pGBT-PH127 are chosen for sequencing.

In our screen of 1.0×10⁶ primary transformants we identify 37 clones which show specific interaction with RAC's PH domain, by activation of the reporters for His auxotrophy and LacZ activity. These clones could be subdivided into 6 different cDNA classes, based on the size of the cDNA insert. Upon sequencing all clones were found to encode human IMPDH type II inclusive of the initiator methionine through to the termination codon of the previously cloned cDNA [Collart, F. R., and Huberman, E. (1988) J. Biol. Chem. 263, 15769-15772].

The interaction requires a complete PH domain as constructs containing either subdomains I-III (amino acids 1-47) or subdomains IV-VI (amino acids 47-127) alone do not show any interaction with IMPDH. The lack of interaction with subdomains IV-VI is significant as this region has previously been shown to interact weakly with the βγ-subunits of heterotrimeric G-proteins. This interaction of IMPDH and RAC's PH domain is however inhibited in the two-hybrid system with constructs containing RAC's kinase domain juxtaposed to RAC's PH domain as occurs in its natural context. This could be due to an intramolecular interaction of RAC's PH domain with itself or another region of RAC. However, inclusion of amino acids between the PH and kinase domains (including the first four amino acids of the kinase domain) didn't inhibit the interaction. We also fuse RAC's PH domain to the GAL4 activation domain to test if it could interact with any of the human RACα GAL4 DNA binding constructs. We can detect no such interaction, indicating that the PH domain does not self associate or form a complex with other regions of the RAC-PK molecule in this system. The inhibition of interaction observed above would thus appear to be due to steric hindrance.

We construct nested amino and carboxyl-terminal deletions of IMPDH to determine the region of the molecule responsible for interaction with RAC's PH domain. This indicates that an almost intact IMPDH molecule is required for the interaction. The amino-terminal boundary of the PH interaction domain is found to lie between amino acids 28 and 70, while the carboxyl-terminal boundary lies between amino acids 427 and 481.

(d) In vitro Binding Studies

To test if IMPDH could interact directly with RAC's PH domain we employ an in vitro binding assay system using GST fusions. GST fusions produced from the plasmids pGEX-2T, pGEX-PH131 and pGEX-IMPDH are expressed in E. coliXL-1 blue cells by induction with 0.1 mM IPTG for 2 hours at 24° C. The fusion proteins are purified as described [Smith, D. B., and Johnson, K. S. (1988) Gene 67, 31-40] except that the cells are lysed in a French Press. The human RACα PH domain (HIS)₆ tagged fusion produced by BI21 (DE3)pLysS cells transformed with pRSET-PHQKKK is expressed and purified as described [UK patent application 9525705.1]. Briefly, cells are induced with 0.2 mM IPTG for 2 hours at 24° C. before harvesting. Cell pellets are lysed in a French Press and the soluble PH domain is purified sequentially on Ni(II) affinity, cation exchange and gel filtration columns. Binding studies are performed using GST fusions (2.5 μg) coupled to glutathione-agarose beads in binding buffer (20 mM phosphate buffer pH 7.2, 150 mM NaCl, 1% Triton X-100, 5 mM DTT) containing 2.5 μg of (HIS)₆-tagged PH domain or baculovirus produced human RACα in a total volume of 100 μl. The samples are incubated at 4° C. for 1-2 hours with agitation every 5 min. The beads are then washed three times with buffer (20 mM phosphate buffer pH 7.2, 150 mM NaCl) before being analysed by SDS-PAGE and stained with coomassie blue R-250 (for (HIS)₆-tagged PH domain binding) or SDS-PAGE followed by Western blot analysis using a human RACα specific antiserum (for human RACα binding) [Jones et al., Op. Cit.]. The secondary antibody is a horseradish peroxidase coupled anti-rabbit antibody (Amersham) which is detected using the ECL method (Amersham) by autoradiography. In this assay we see that the (HIS)₆-tagged PH domain can bind to the GST-IMPDH fusion but not to GST alone.

We also employ this assay system to test if full-length baculovirus purified human RACα could directly interact with IMPDH. Full-length human RACα is expressed and purified from the baculovirus system as described [see UK patent application 9525702.8]. Briefly, a baculovirus is constructed by co-transfection of Sf9 cells with pVL1392-hRACα and wild-type baculovirus AcMNPV DNA and purified by limiting dilution and detected by dot-blot hybridisation. The purified virus is used to produced human RACα in Sf9 cells. The human RACα is purified by sequential anion exchange, phospho-cellulose and gel filtration chromatography. Here we see specific interaction of the full-length RAC-PK molecule with GST-IMPDH and not GST alone or the GST-PH fusion.

(e) In vivo Pull-down Assay

Using GST-IMPDH in a pull-down assay with MCF-7 cell extracts we see a specific association of human RACα with the GST-IMPDH and not with GST. MCF-7 cells are lysed in buffer (50 mM Tris-HCl pH 8.0, 120 mM NaCl, 1% NP-40, 1 mM EDTA, 1 mM EGTA, 30 mM pNPP, 25 mM β-glycerol phosphate, 15 mM PPi, 25 mM NaF, 1 mM vanadate, 20 μM PAO, 1 mM benzamidine, 0.1 mM PMSF) using twelve strokes of a dounce homogeniser. Soluble protein from the supernatant of lysates centrifuged at 14,000×g for 15 min at 4° C. are added to GST, GST-PH or GST-IMPDH protein (5 μg) attached to glutathione beads and incubated at 4° C. for 2 hours with continuous agitation. The beads are then washed four times with lysis buffer before being analysed by Western blotting as described above with the human RACα specific antiserum [Jones et al., Op. Cit.; (1991) Cell Reg. 2, 1001-1009] or an IMPDH specific antiserum [Collart, F. R., and Huberman, E. (1988) J. Biol. Chem. 263, 15769-15772]. We could also perform the converse experiment, pulling down IMPDH from cell lysates using the GST-PH domain fusion protein. Thus we have shown the existence of an association between human RACα and human IMPDH type II in three heterologous systems.

(f) Enzyme Assays

We then assay the effect of this interaction on the activity of IMPDH. The addition of soluble PH domain as a GST fusion produced an activation of IMPDH compared to the addition of GST alone. Assays for IMPDH activity are performed essentially as described [Antonio, L. C., and Wu, J. C. (1994) Biochem. 33, 1753-1759] monitoring the production of XMP by absorbance at 286 nm. The IMPDH is produced as a GST fusion purified on glutathione beads and then eluted as soluble protein with reduced glutathione. IMPDH activity is tested in the presence of either soluble GST (produced from pGEX-2T) or PH domain (produced from pGEX-PH131) at a molar ratio of IMPDH to GST/PH domain of 1:5. RAC kinase assays with the baculovirus produced human RACα using various substrates (myelin basic protein, GST or GST-IMPDH) are performed essentially as described [Jones et al., Op. Cit.]. When IMPDH is tested in RAC kinase assays using the baculovirus produced human RACα to see if it is a substrate we could detect no significant phosphorylation of the IMPDH.

### EXAMPLE 2

### Inhibition of GSK3

Two major kinases known to be involved in regulating the insulin-dependent signalling pathways are MAPKAP kinase-1 and p70^{S6K}. These kinases are respectively inhibited by PD98059 and rapamycin. Both of these agents fail to inhibit phosphorylation of GSK3, suggesting that the kinase responsible for GSK3 inactivation is not MAPKAP kinase-1 or p70^{S6K}.

L6 myotubes are incubated with both compounds and the stimulated with insulin, as follows. Monolayers of L6 cells are grown in 6 cm petri dishes to the stage of myotubes, deprived of serum overnight and then incubated for 1 hour in 20 mMHepes/NaOH (pH 7.4) 0.14M NaCl/5 mM KCl/2.5 mM MgSO₄/1 mM CaCl₂/25 mM glucose (HBS buffer), in the presence or absence of 50 μM PD98059 or 100 μM LY294002. 2 mM 8-Br-cAMP or 0.1 μM rapamycin, when added, are included for the last 15 min. The cells are stimulated for 5 min. with 0.1 μM insulin, or for time courses of up to 10 min. The medium is removed by aspiration, the cells placed on ice and lysed in 0.2 ml of ice-cold buffer A [50 mM Tris-HCl (pH7.5, 20° C.)/1 mM EDTA/1 mM EGTA/1% (w/v) Triton X-100/1 mM sodium orthovanadate/10 mM sodium glycerophosphate/50 mM sodium fluoride/5 mM sodium pyrophosphate/2 μM microcystin/0.1% (v/v) 2-mercaptoethanol/leupeptin 4 μg/ml, 1 mM benzamidine, 1 mM phenylmethane sulphonyl fluoride, 30 μg/ml aprotinin, 30 μg/ml antipain, 10 μg/ml pepstatin.

Precipitation of p42^{MAPK}, MAPKAP kinase-1 or GSK3 from the cell lysates by immunoprecipitation and analysis of their activity with specific protein or peptide substrates (Cross et al., (1994) Biochem. J. 303, 21-26) shows that insulin inactivation of GSK3 is not affected by agents (2 mM 8-Br-cAMP or 0.1 μM rapamycin) which inhibit classical MAP kinase or p70^{S6K} signalling pathways.

In order to identify the kinase which inhibits GSK3 in the presence of rapamycin and PD98059, cells are lysed as above and the cell lysates (0.3 mg) chromatographed on a 5×0.16 cm column of Mono Q (Sutherland et al. , (1994) FEBS Lett. 338, 37-42) except that the buffer additionally contains 1 mM EGTA, 0.1 mM sodium orthovanadate and 0.5% (w/v) Triton X-100.

The fractions (0.05 ml) assayed with the synthetic peptide GRPRTSSFAEG, which corresponds to the sequence of GSK3 surrounding the serine (bold type) whose phosphorylation triggers the inactivation of GSK 3α (Ser²¹) and GSK3β (Ser⁹). Three peaks of activity which result in phosphorylation of this peptide are eluted. These peaks are absent if insulin stimulation is not given, or if cells are incubated with 0.1 μM wortmannin prior to insulin stimulation. The inactivating effect of insulin on GSK3 is known to be prevented by administering this concentration of wortmannin, or 100 μM LY294002, a structurally unrelated inhibitor of PI-3K.

All three peaks of phosphorylating activity can be immunoprecipitated with an anti-RAC antibody using a polyclonal rabbit antiserum directed against the peptide FPQFSYSASSTA raised by injecting rabbits subcutaneously with the peptide and purified by precipitation using 50% (NH₄)₂SO₄ followed by affinity chromatography on RAC-peptide coupled Affigel® 10 column (Bio-Rad).

In contrast, immunoprecipitating with an anti-MAPKAP kinase-1 antibody fails to deplete any peptide phosphorylating activity from the Mono Q column.

In order to determine that complete GSK3 can be inactivated by RAC, GSK3α and GSK3β are partially purified from rabbit skeletal muscle (Sutherland et al. , Op. Cit.) and assayed with a specific peptide substrate (Cross et al., Op. Cit.). Each GSK3 isoform is diluted to 15 U/ml and GSK3 activity measured after incubation for 20 min with MgATP in the presence or absence of RAC. The incubation is the made 2 mM in EDTA to stop the kinase reaction, incubated for 20 min with 5 mU/ml PP2A₁ to reactivate GSK3, made 2 μM in okadaic acid to inactivate PP2A₁ and then assayed for GSK3 activity.

In the absence of RAC, GSK3 is stably active throughout the experiment. Otherwise, however, RAC-PK successfully inhibited GSK3 activity and this inactivation was sensitive to PP2A₁, which restored GSK3 activity. The absence of insulin stimulation, the presence of wortmannin or the disruption of RAC-PK immunoprecipitation by incubation of the anti-RAC antibody with the peptide immunogen all result in a lack of GSK3 inactivation in this experiment.

### EXAMPLE 3

To determine if RAC's kinase domain with its carboxyl-terminal extension could interact with other proteins we fused it to the GAL4 DNA binding domain and screened a HeLa cDNA library fused to the GAL4 transcriptional activation domain in the yeast reporter strain HF7c following the procedure of Example 1. Briefly, amino acids 147-480 of RACα are fused in frame to GST in the expression vector pGEX-2T (see Example 1). Appropriate BamHI-EcoRI fragment is then subcloned into the PstI-XbaI sites of yeast vector pPC62 (Dr. D. Nathans) using PstI-BamHI and EcoRI-XbaI linkers in order to create a GAL4 DNA binding domain-RAC kinase and C-terminal domain fusion. XhoI-XbaI fragments are then subcloned into pGBT9 (Clontech). The HeLa S3 MATCHMAKER cDNA library is used as before.

In our screen of 1.5×10⁶ primary transformants we identify seven clones which show specific interaction with RAC's kinase domain plus the carboxyl-terminal extension, by activation of the reporters for His auxotrophy and LacZ activity. A detailed analysis of the specific region of RAC-PK that the clone interacts with shows that the carboxyl-terminal 69 amino acids are all that is required to confer the interaction. We thus denote this molecule CTBP, for Carboxyl-Terminal Binding Protein. None of the clones shows an interaction with the kinase domain alone. This interaction is seen in all constructs containing the carboxyl-terminal extension including a full-length RAC-PK construct, indicating that the interaction is not inhibited by another region of the RAC-PK molecule. Interestingly, the C-terminal domain of RAC-PK is phosphorylated in response to insulin activation, suggesting a role for CTBP as a modulator of insulin action.

All seven specific interacting clones contain identical cDNA inserts of 1.3 kb in length with an ALU repeat of ˜300 nt at the 3′ end (SEQ ID No. 1). Searches of the Gene-EMBL nucleotide database using the cDNA sequence without the ALU repeat with the FASTA programme (GCG Package) identify no significant homologies. The deduced amino acid sequence of the CTBP cDNA produces a short 47 residue polypeptide rich in alanines (21%) and arginines (21%). Searches of the PIR, Swiss-Prot, and GP protein databases using FASTA (GCG Package) and the Gene-EMBL nucleotide database using TFASTA (GCG Package) reveal no significant homologies to the CTBP protein sequence.

The sequence of CTBP identified is believed to represent the 3′ end of the complete CTBP molecule.

To test if the novel protein CTBP could interact directly with RAC-PK we employ an in vitro binding assay system using GST fusions, as described in Example 1. In this assay we see specific interaction of the full-length RAC, produced in the baculovirus system with GST-CTBP and not GST alone.

To test if this interaction occurs in vivo we employ a pull-down assay using the GST-CTBP fusion protein and MCF-7 cell extracts, as described in Example 1. Here we see the specific association of the MCF-7 RACα with the GST-CTBP but not GST alone.

1. Use of RAC protein kinase, or a modulator thereof except wortmannin, in the manufacture of a composition for use in the treatment of abnormalities in cellular proliferation.

2. Use of RAC protein kinase, or a modulator thereof except vanadate, in the manufacture of a composition for use in the treatment of diseases involving an anomaly in insulin response.

3. Use of claim 2, wherein the disease involves an anomaly in glycogen metabolism.

4. A method for screening potential modulators of insulin-mediated intracellular signalling comprising the steps of:
(a) incubating RAC-PK or a functional fragment thereof with the compound to be screened; and
(b) detecting interaction between the compound and RAC-PK or its functional fragment.

5. Method of claim 4 wherein the RAC-PK fragment is selected from the PH domain, the catalytic domain and the C-terminal domain.

6. A modulator of insulin-mediated intracellular signalling when identified by a method of claim 4.

7. A modulator of claim 6 which is selected from the group consisting of IMPDH, GSK-3 and a polypeptide comprising the sequence set forth in SEQ ID No. 1.

8. A kit comprising:
RAC, or a functional fragment thereof;
means for incubating RAC-PK or its functional fragment with a compound to be screened for its potential to modulate insulin-mediated intracellular signalling; and
means for detecting an interaction between RAC-PK or its functional fragment and the compound.

9. RAC-PK having one or both of the mutations T308->D and S473->D.

10. RAC-PK of claim 9 for use in a method for screening potential modulators of insulin-mediated intracellular signalling comprising the steps of:
(a) incubating RAC-PK or a functional fragment thereof with the compound to be screened; and
(b) detecting interaction between the compound and RAC-PK or its functional fragment.

11. A method of screening for compounds that increase or decrease the kinase activity of RAC-PK comprising:
(a) incubating a polypeptide comprising amino acids 1-480 of SEQ ID. No.3, or amino acids 1-411 of SEQ ID. No. 3, or amino acids 147-411 of SEQ ID. No.3, or amino acids 147-480 of SEQ ID. No.3, wherein said polypeptide interacts with GSK3, with a compound to be screened and with a substrate capable of being phosphorylated by RAC-PK;
(b) measuring the amount of phosphorylated serine or threonine residues in said substrate; and
(c) selecting said compound if the amount of phosphorylated serine or threonine residues in said substrate is larger or smaller in the presence of said compound than in the absence of said compound.

12. A method of claim 11 wherein amino acid 308 of SEQ ID. No. 3 is an acidic amino acid residue, or amino acid 473 of SEQ ID. No. 3 is an acidic amino acid residue, or both amino acid 308 of SEQ ID. No. 3 and amino acid 473 of SEQ ID. No. 3 are acidic amino acid residues.

13. A method of claim 12 wherein any said amino acid residue is an aspartic acid residue.

14. A method of claim 11 wherein the substrate for measuring the kinase activity comprises the sequence GRPRTSSFAEG.

15. A method for screening inhibitors of RAC-PK comprising:
(a) incubating a polypeptide comprising amino acids 1-480 of SEQ ID. No.3, or amino acids 1-127 of SEQ ID. No. 3, wherein said polypeptide interacts with IMPDH, with a compound to be screened and with a substrate capable of being phosphorylated by RAC-PK;
(b) measuring the amount of phosphorylated serine or threonine residues in said substrate; and;
(c) selecting said compound if the amount of phosphorylated serine or threonine residues in said substrate is larger or smaller in the presence of said compound than in the absence of said compound.
